(19) United States
(12) Patent Application Publication
(10) Pub. No.: US 2012/0232596 A1
(43) Pub. Date: Sep. 13, 2012

(54) MULTI-ADJUSTABLE PLATE FOR OSTEOTOMY

(76) Inventor: Christiano Hossri Ribeiro, Sao Jose dos Campos (BR)

(21) Appl. No.: 13/508,253

(22) PCT Filed: Nov. 11, 2010
(86) PCT No.: PCT/IB10/55131

§ 371 Date: May 23, 2012

(30) Foreign Application Priority Data
Nov. 5, 2009 (CH) 01704/09

(51) Int. Cl. A61B 17/80

(52) U.S. Cl. 606/289

(57) ABSTRACT

The invention relates to a mechanical device intended for correcting a malformation of the bones of the body. The device is continuously adjustable, making the surgeon's work easier and reducing surgery time, thus reducing the risk of infection and, above all, guaranteeing the desired precision for surgery. The device comprises a top plate (1) suitable for sliding over a bottom plate (2) by means of a guide (7). Said plates can be screwed on either side of an angled notch made in the bone. The plates comprise respectively top (5) and bottom (6) bearings of the notch of the bone. Said bearings form an acute-angle bevel which substantially matches the surfaces of the notch. One of the plates is provided with an adjustment screw (4) for continuous adjustment of the relative positions of the plates. The device also comprises a locking screw (3) suitable for maintaining the open position of the device and, consequently, the final angle of the notch of the bone in the desired position. The Top bearing of the notch of the bone having angular adjustment is suitable for correcting the angle of the bevelled surface of the bearing, in particular the tibial slope, by means of a tapered screw (8) adjustment an angular space in the bearing.

The invention relates to a mechanical device developed to correct a misalignment malformation of the body bones. The device consists of a plate of osteotomy, allowing the millimetre-length, self-adjustment correction, regulating the opening of the corner fixed as a preliminary, also allowing the adjustment of the slope.

### INTRODUCTION

The osteotomies are processes used since many years to correct certain deformations of the bones. These processes can be applied to various bones, as femur, tibia, calcaneus, metatarsus, radius, humerus, etc. The malformation of the bone occurs for several reasons, by causing for example a misalignment of the leg, by forming an angle between the femur and the tibia, having a curved appearance when it is observed the front one (FIG. 1)

The osteotomies carried out around the knee (femur and tibia) are very popular, and underwent an evolution as for their fixing. At the beginning of the 20th century, osteotomy used to be carried out by using the plaster like instrument of fixing. Thereafter, other synthesis materials emerged, like wires, screws and plates, getting more stability and safety to the process. In 2002, the Plate of Puddu for fixing of the osteotomy with opening corner was made known in the entire world. From there, new plates appeared on the market. The plates work primarily like an instrument of fixing of the osteotomy, whereas other stages of the surgery are carried out by other instruments. While proceeding in this manner, it is frequent to observe complications inherent in the surgical act and in the method of fixing used with the devices of the prior art:

1) Excessive time of the surgery, due to various stages (surgical times) to be carried out during the operation:
a) Passage of the wire-guide, cutting out of the bone, opening of the corner with tuning fork, choice of the adequate plate, withdrawal of the tuning fork, fixing of the plate. Because of this great technical difficulty, few surgeons are entitled to carry out such a procedure.
b) Risk of infection increased.
c) Risk of neurological lesion increased.
d) More bleeding during the operation.

2) Fracture of the side cortex of the tibia, one of the most feared and frequent complication in the osteotomy of the tibia with opening corner. This occurs when the notch of the osteotomy reaches the side cortex of the tibia, causing fracture and subluxation of the bone.

3) Fracture of the articular surface. Occurs when the height and the slope of the notch of the osteotomy is made in a wrong way.

4) Hypo and hypercorrection. Complication directly related to the used plate, because they are not all the types of plates which allow a millimetre-length fitting of the opening corner; this complication can also come from an inadequate fixing, or a wrong preoperative planning.

5) Deterioration of the tibial slope. Consist of the deterioration of the slope of the tibia in the sagittal plan, characterized clinically by the loss of the extension of the knee.

### Disadvantages of the Devices of the Prior Art and Observed Problems

The devices usually used to correct malformations of the bones comprise two plates which move one on the other, in the axial direction, in general guided to maintain their alignment (see FIG. 2).

There are systems which consist of a plate and of a part of spacing gauged (a corner) which will be introduced into the bone with predefined measurement fixes, which corresponds to the desired aperture.

The plates used currently for the osteotomy do not have a millimetre-length system of correction, which requires a set of plates differently numbered according to the correction to be made. The exact wished measurement was not found, because the measurements provided by the set measure from 2.5 in 2.5 mm.

Moreover, the plates are used only in final stage of the surgery, with an only aim of fixing; they do not intervene in the adjustment of the tibial slope.

With the devices of prior art quoted above, which are pre-gauged, the surgical procedure is the same one, in the case of correction of the tibia for angular malformation, namely: one cuts out a notch in angle or notch in the tibia, very close to the knee. It is introduced then a tuning fork (graduated corner, to see FIG. 8) to facilitate the opening of the bone until obtaining the desired angle, by using a hammer to insert the corner in the bone. Once obtained the ideal opening, it places the plate with intercalated part dimensioned as a preliminary. Finally, the plate is fixed, then it withdraws the tuning fork (graduated corner) with precaution, through a very reduced workspace.

By withdrawing the tuning fork, the fastening screws tight then are blocked.

With all the existing devices on the market, it is necessary to notch very deeply the bone, which, depend on the angle of correction, presents a great risk of shearing of the bone, because of necessary forces important to introduce the precalibre corner, which can cause:

1) A trauma in the side cortical;

2) Surface trauma of the arteries, since when the surgery is carried out, blood will stagnate in the arteries without being able to circulate for a period of 2 hours to the maximum, which is the safety limit;

3) Excessive duration of the surgery, which increases the risks of infection.

4) Neurovascular lesion.

This last point constitutes a factor of complication which would prolong the surgery, being able to involve the effects quoted above.

Another problem of the devices currently used is the lack of guarantee as for obtaining the opening of the exact desired value. For example, in the case of the device with teeth of saw, known under the name of “Plate of Anthony” (FIG. 3), where the top plate is fixed at the top of the notch, while the bottom plate is fixed below the notch (FIG. 2), it may be that the value of the desired opening corresponds to a position where the teeth are not encased. In this stage, the surgeon must decide if he will be satisfied with an opening lower than the wished, or on the contrary, to choose a top opening. (Problem: plate without millimetre-length fitting, of little precision).

The same problem emerges for the systems to intercalated part of fixed, interchangeable size (FIG. 4) known under the name of Plate of Puddu already quoted, having a part to be chosen among tens of options which the kit comprises, part which is used as bearing for the cutting of the tibia. Problem: much time devoted to seek and choose the intercalated part having required exact dimension, involving the increase in the time of the surgery with more exposure to the risks already mentioned.

### The Requested Invention

The goal of this patent application is to work out a mechanical device having for objective to correct a malformation of the body bones, with continuous adjustment, making it possible to facilitate the work of the surgeon by decreasing the time of the surgery, to thus decrease the risks of infection, and mainly to guarantee the precision desired for the surgery, while not presenting the disadvantages quoted above.

The solution making it possible to achieve this goal is contained in the characteristics of the independent claim number 1.

The invention makes it possible to make a notch of restricted angle, quite less than the angle required by the devices of prior art.

The invention consists of two Plates, the Bottom plate and the Top plate, and the opening or closing is regulated via a screw, which according to the direction according to which one turns it, allows to draw aside or to bring closer the two Plates.

This new concept of Plates allows the introduction of the Top bearing and Bottom bearing of Tibia into the restricted space of the notch, to tighten at the same time the screws fastening of the Top plate and Bottom plate, then to carry out the precise adjustment of the spacing of the Plates.

The use of this system with continuous adjustment, unresolved of continuity, is by no means limited to the bones of the legs, but can be used on all the bones of the body, either with an aim of correction, or like permanent prosthesis.

### Advantages of the Invention

1) Synthetic material device, allowing the self-adjustment millimetre-length correction, in a single device.

2) Fixing allows immediately after the realization of the notch in the bone, thus getting a safety increased as for the risk of side cortical fracture and intra-articular fracture, and reducing the duration of the surgery, thanks to its simple technique.

3) Less risk of trauma of articular surface, because the plate is fixed during the opening of the osteotomy.

4) Influence in the final result significantly, because it makes possible to carry out any necessary correction.

5) This device allows the adjustment and the modification of the deviation tibiale, thanks to the system of correction of the tibial slope.

6) Assured precision thanks to the notch reduced in the tibia, due to the progressive adjustment of its opening until exact desired point.

### DETAILED DESCRIPTION OF THE INVENTION

The invention is now described in detail while also referring to the additional figures. The device comprises eight principal parts (see FIG. 5):

1) Top plate

2) Bottom plate

3) Locking screw

4) Adjustment screw

5) Top bearing of the notch of the tibia with angular adjustment

6) Bottom bearing of the notch of the tibia

7) Guidance system in “dovetail”

8) Tapered screw of adjustment of the tibial slope

The plates can be introduced into the notch of reduced size made in the femur as represented on the FIG. 6.

The screws fastening of the top plate and bottom plate once put in position can be screwed on both sides of the notch. Then, the top bearing and bottom bearing of the notch of the tibia being in position, the spacing of the plates can start via adjustment screw (4)

By considering that the top plate and bottom plate are guided between them by a dovetail guidance system (7), they remain aligned and the continuous adjustable tangent makes it possible to draw aside them gradually.

The measurement of the opening can be controlled millimetrically with utmost precision until obtaining the desired position.

Once confirmed the wished position, one can block the position obtained using locking screw (3) to maintain the exact position of the space between the two plates, it results the aperture from it from the notch of the femur to the exact desired value.

Locking screw (3) has as a function to block the top plate on the bottom plate, thus guaranteeing the maintenance of the desired position.

After this blocking, the fastening screws of the top plate and bottom plate (FIG. 6) can be tight and blocked definitively.

Lastly, if it proves to be necessary to correct the angle of the top bearing of the notch of the tibia with angular adjustment, one can do it using a tapered screw (8), which allows the opening of an existing angular space in the top plate thus correcting the tibial slope, as represented on the FIG. 7.

Once this correction carried out, with all fastening screws blocked, the operation of correction of malformation is finished.

### Legend of the Figures

FIG. 1

1.1)—Femur

1.2)—Tibia.

FIG. 2

2.1)—Notch in angle of the tibia.

2.2)—Top plate.

2.3)—Bottom plate.

2.4)—Fastening screw.

FIG. 3.

3.1)—Top plate

3.2)—Bottom plate

3.3)—Top bearing of the notch of the tibia.

3.4)—Bottom bearing of the notch of the tibia.

FIG. 4

4.1)—Plate

4.2)—Interchangeable corner of bearing of the notch of the tibia.

4.3)—Set of interchangeable corners.

FIG. 5

1)—Top plate

2)—Bottom plate

3)—Locking screw

4)—Adjustment screw

5)—Top bearing of the notch of the tibia with angular adjustment

6)—Bottom bearing of the notch of the tibia.

7)—Guide of the type “dovetail”.

8)—Tapered screw of adjustment of the tibial slope.

FIG. 6

6.1)—Top fastening screw.

6.2)—Top plate.

6.3)—Lower fastening screw.

6.4)—Bottom plate.

6.5)—Femur.

6.6)—Tibia.

FIG. 7

7.1)—Tapered screw for the angular opening.

7.2)—Top bearing of the notch of the tibia with angular adjustment.

7.3)—Top plate.

FIG. 8

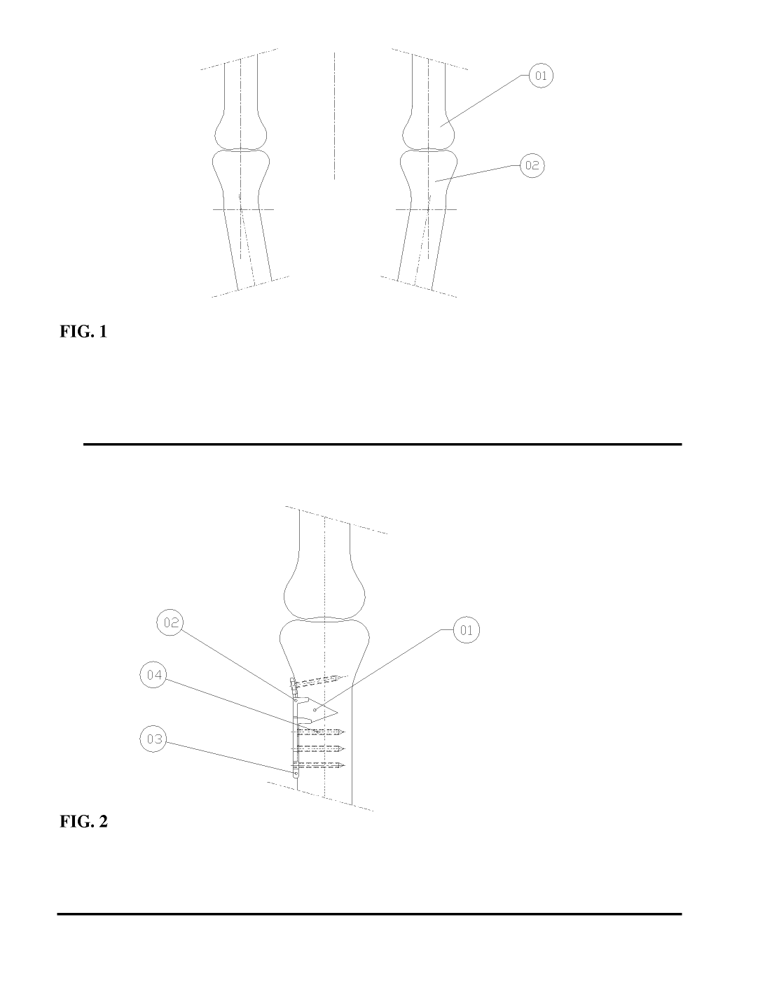

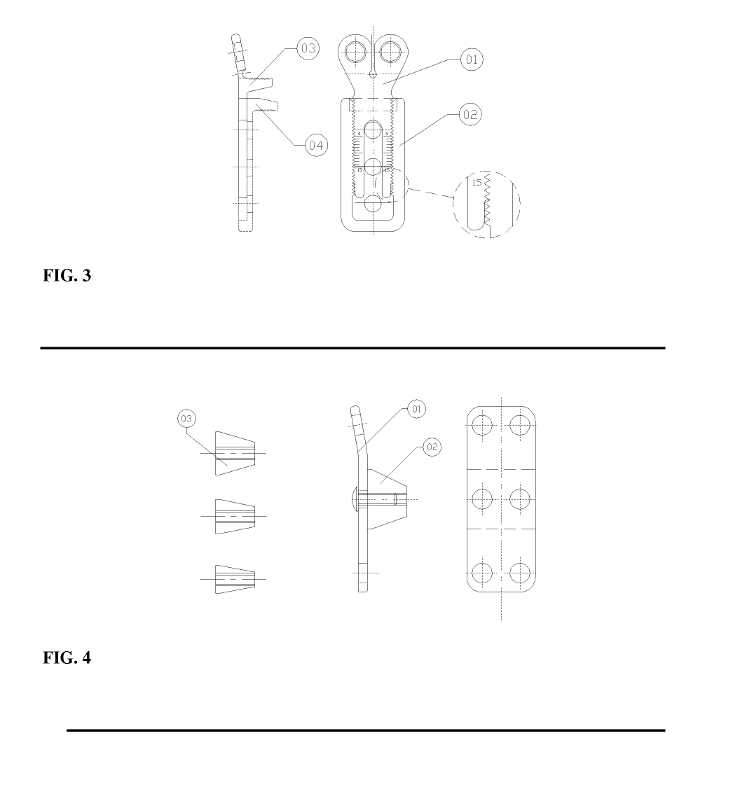

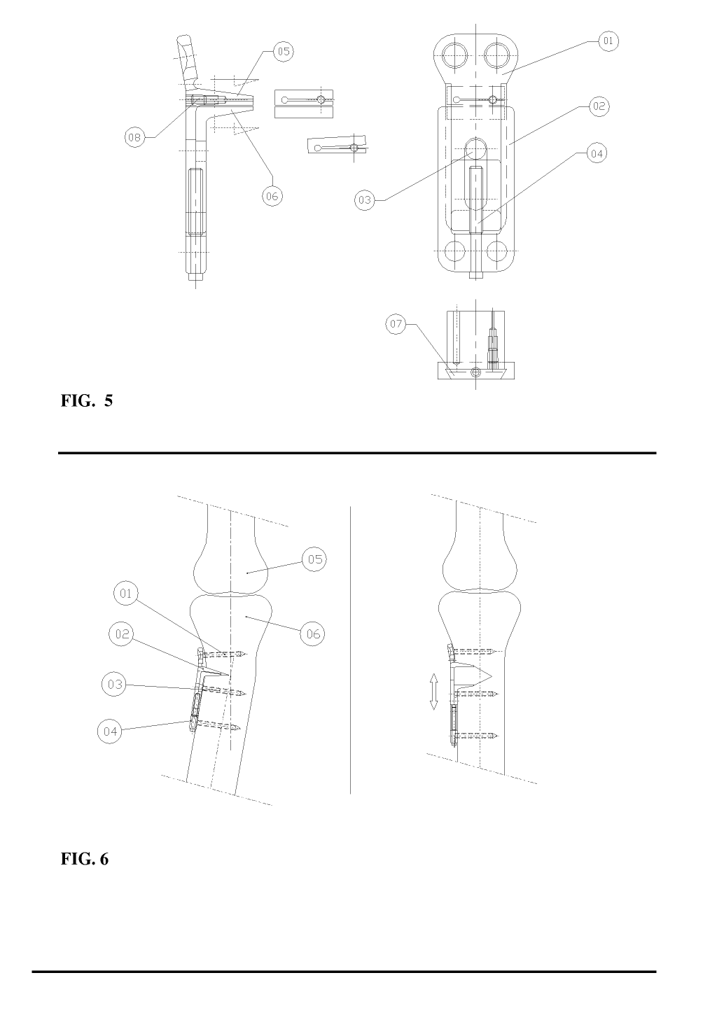

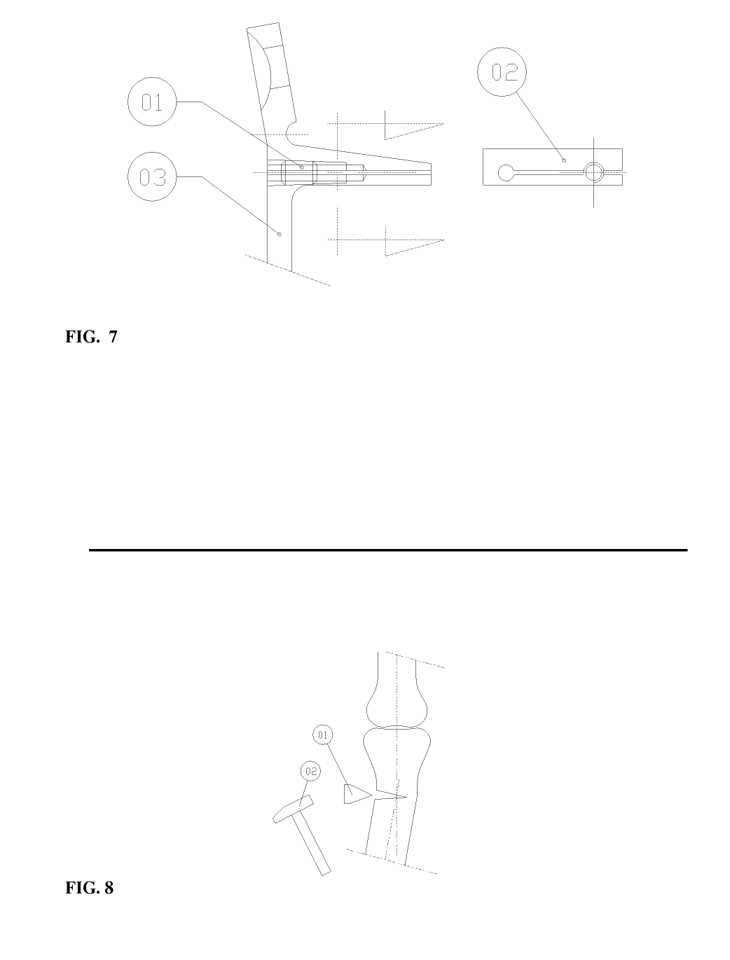

8.1)—Tuning fork (corner)

8.2)—Hammer for the opening of the notch by using the tuning fork.

1. Mechanical device having as goal to correct a malformation of the bones of the body, with continuous adjustment, allowing to facilitate the work of the surgeon by decreasing the time of the surgery, to thus decrease the risks of infection, and mainly to guarantee the desired precision for the surgery, wherein a top plate (1) is able to slide on a bottom plate (2) by using a guide (7); these plates being able to be screwed on both sides of a notch in an angle made in the bone; the plates comprising a top bearing (5) and a bottom bearing (6) respectively of the notch of the bone; these bearings being directed according to a direction appreciably perpendicular with the plates to be able to be introduced into the notch of the bone; these bearings having moreover a form of bevel of acute angle so as to marry the faces of the notch appreciably; wherein moreover in what one of the plates is equipped with an adjustment screw (4) continuous of the position of the plates between them; the device comprising moreover a locking screw (3) allowing to maintain the position of opening of the device and consequently the final angle of the notch of the bone in the desired position.

2. Device according to claim 1, wherein the guide (7) makes possible the plates to slide one on the other comprises a profile in the shape of dovetail.

3. Device according to one of the claim 1 or 2, wherein the top bearing of the notch of the bone comprises an angular adjustment making it possible to correct the angle of the bevelled face of the bearing, in particular the tibial slope.

4. Device according to claim 3, wherein the angular adjustment is obtained by using a tapered screw (8) who allows the opening of an existing angular space in the top plate.

5. Device according to one of the claim 1 or 2, wherein the bottom bearing of the notch of the bone comprises an angular adjustment making possible to correct the angle of the bevelled face of the bearing, in particular the tibial slope.

6. Device according to claim 5, wherein the angular adjustment is obtained by using a tapered screw who allows the opening of an existing angular space in the bottom plate.
